(19) United States
(12) Patent Application Publication
(10) Pub. No.: US 2026/0090784 A1
(43) Pub. Date: Apr. 2, 2026

(54) Electronic Stethoscope

(71) Applicant: Murata Manufacturing Co., Ltd., Nagaokakyo-shi (JP)

(72) Inventors: Hirofumi TSUCHIMOTO, Nagaokakyo-shi (JP); Masatoshi SUNAYAMA, Nagaokakyo-shi (JP)

(21) Appl. No.: 19/341,368

(22) Filed: Sep. 26, 2025

(30) Foreign Application Priority Data
Sep. 27, 2024 (JP) 2024-169043

(51) Int. Cl. A61B 7/04; A61B 90/00

(52) U.S. Cl. CPC A61B 7/04; A61B 90/08; A61B 2090/0807; A61B 2562/0204

(57) ABSTRACT

An electronic stethoscope includes a casing, a sound sensor that is provided at the casing and that obtains a sound of an organism and converts the sound of the organism into an electrical signal, light-emitting elements that are provided in the casing and that have respective light-emitting surfaces that emit light beams; and a light-guiding member that guides the light beams of the light-emitting elements to outside of the casing. The light-guiding member has an inner surface that includes light-receiving portions that face the light-emitting surfaces and upon which the light beams L of the light-emitting elements are incident, and an outer surface that is exposed to the outside of the casing by extending along an outer peripheral surface of the casing, and that radiates to the outside of the casing the light beams that are incident upon the outer surface through the light-receiving portions.

### TECHNICAL FIELD

The present disclosure relates to an electronic stethoscope.

### BACKGROUND ART

For example, Patent Document 1 describes an electronic stethoscope that emits light for notifying a user of predetermined information. The electronic stethoscope described in Patent Document 1 is an electronic stethoscope including a plurality of light-emitting elements that are disposed side by side along the same circumference. When any one of the plurality of light-emitting elements emits light, the user is notified of a direction of existence of a sound source (for example, the heart) with respect to the electronic stethoscope.

### CITATION LIST

### Patent Document

[Patent Document 1] International Publication No. 2023/136175

### SUMMARY OF INVENTION

### Technical Problem

As with the electronic stethoscope described in Patent Document 1, when a user is notified of predetermined information by light emission by a plurality of light-emitting elements, the plurality of light-emitting elements consume a large amount of electric power and thus a long-time operation of the electronic stethoscope may be prevented.

To solve this problem, the number of light-emitting elements may be decreased. However, in this case, the transmissibility of the information to the user may be reduced. For example, when any of the light-emitting elements covered by any of the fingers of the user emits light, the user does not notice the light emission thereof and thus cannot obtain information based on the light emission.

Accordingly, an object of the present disclosure is to, in an electronic stethoscope where light-emitting elements emit light for notifying a user of predetermined information, reliably notify the user of the predetermined information by using a small number of light-emitting elements.

### Solution to Problem

To solve the above-described technical problem, according to an aspect of the present disclosure, there is provided an electronic stethoscope including a casing that has a first end surface that faces an organism when being used, a second end surface that is situated on a side opposite to the first end surface, and an outer peripheral surface that connects the first end surface and the second end surface to each other;
a sound sensor that is provided at the casing and that obtains a sound of the organism and converts the sound of the organism into an electrical signal;
a light-emitting element that is provided in the casing and that has a light-emitting surface that emits light; and
a light-guiding member that guides the light of the light-emitting element to outside of the casing,
in which the light-guiding member has
   an inner surface that includes a light-receiving portion that faces the light-emitting surface of the light-emitting element and upon which the light of the light-emitting element is incident, and
   an outer surface that is exposed to the outside of the casing by extending along the outer peripheral surface of the casing when viewed in a facing direction in which the first end surface and the second end surface face each other, and that radiates to the outside of the casing the light that is incident upon the outer surface through the light-receiving portion.

### Advantageous Effects of Invention

According to the present disclosure, the electronic stethoscope where the light-emitting elements emit light for notifying a user of predetermined information can reliably notify the user of the predetermined information by using a small number of light-emitting elements.

### BRIEF DESCRIPTION OF DRAWINGS

FIG. 1 is a perspective view of an electronic stethoscope according to a first embodiment of the present disclosure.

FIG. 2 is a side view of the electronic stethoscope according to the first embodiment.

FIG. 3 is an exploded perspective view of the electronic stethoscope according to the first embodiment.

FIG. 4 is a block diagram of an electronic stethoscope including the electronic stethoscope according to the first embodiment.

FIG. 5 is a perspective view of light-emitting elements and a light-guiding member provided on a circuit board in the electronic stethoscope according to the first embodiment.

FIG. 6 is a cross-sectional view of the light-emitting elements and the light-guiding member in the electronic stethoscope according to the first embodiment.

FIG. 7 illustrates relationships between an inclination angle of a light-emitting element and a radiation angle range of light from an outer peripheral surface of the light-guiding member.

FIG. 8 is a cross-sectional view of light-emitting elements and a light-guiding member in an electronic stethoscope according to a second embodiment.

FIG. 9 is a cross-sectional view of light-emitting elements and a light-guiding member in an electronic stethoscope according to a third embodiment.

FIG. 10 is a cross-sectional view of light-emitting elements and a light-guiding member in an electronic stethoscope according to a fourth embodiment.

FIG. 11 is a cross-sectional view of light-emitting elements and light-guiding members in an electronic stethoscope according to a different embodiment.

FIG. 12 is a perspective view of the electronic stethoscope according to the first embodiment with a light-shielding cover being attached thereto.

FIG. 13 is a top view of the electronic stethoscope according to the first embodiment with the light-shielding cover being attached thereto.

### DESCRIPTION OF EMBODIMENTS

Embodiments of the present disclosure will be described below with reference to the drawings.

### First Embodiment

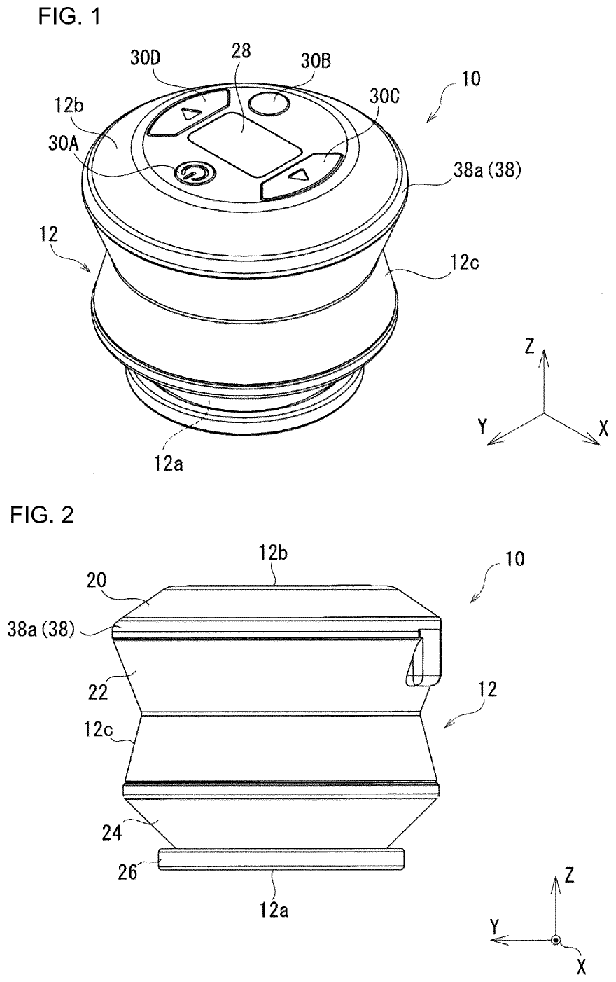

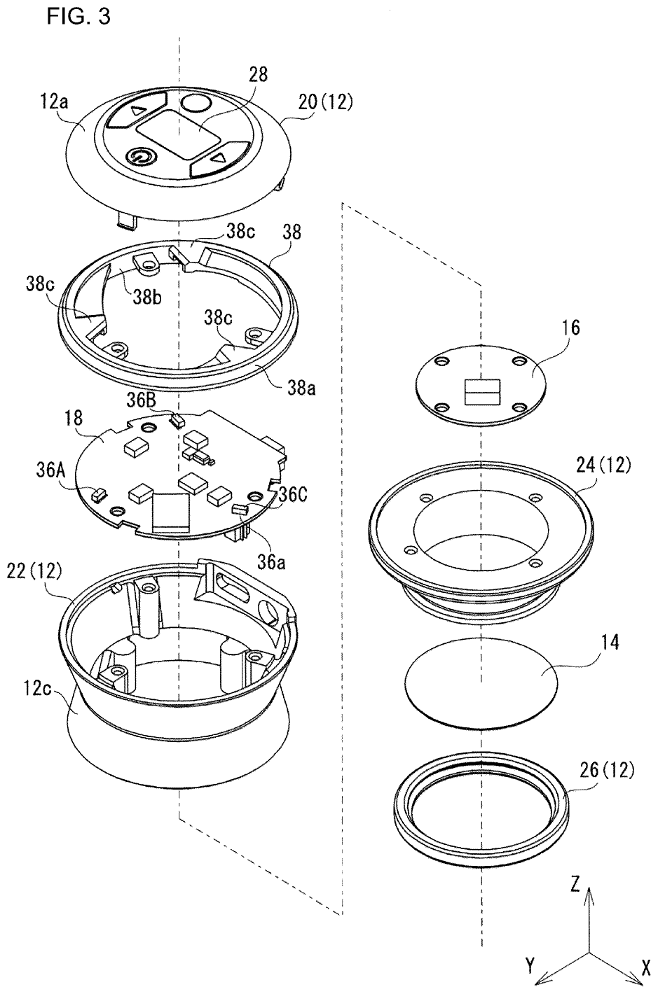

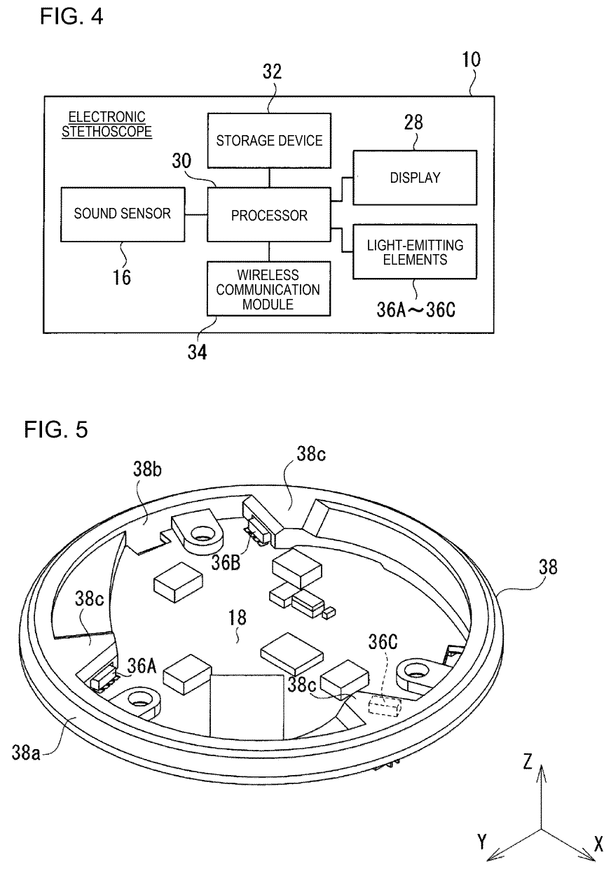

FIG. 1 is a perspective view of an electronic stethoscope according to a first embodiment of the present disclosure. FIG. 2 is a side view of the electronic stethoscope according to the first embodiment. FIG. 3 is an exploded perspective view of the electronic stethoscope according to the first embodiment. FIG. 4 is a block diagram of an electronic stethoscope including the electronic stethoscope according to the first embodiment.

It should be noted that an X-Y-Z orthogonal coordinate system shown in the figures is provided for making it easier to understand embodiments of the present disclosure, and does not limit the embodiments. It should be noted that an X axis direction indicates a width direction of the electronic stethoscope, a Y axis direction indicates a depth direction, and a Z axis direction indicates a thickness direction. The Z axis direction is a direction in which the electronic stethoscope contacts an organism.

An electronic stethoscope 10 according to the first embodiment shown in FIG. 1 is an electronic device that, being in contact with, for example, an organism such as a human being, collects the sound of the organism that is produced by the organism. As shown in FIGS. 1 to 4, the electronic stethoscope 10 includes a casing 12, a diaphragm 14 that faces and comes into contact with an organism, a sound sensor 16 that receives vibration propagated from the diaphragm 14 and that converts the vibration into an electrical signal, and a circuit board 18. It should be noted that structural elements provided in the casing 12 are not limited thereto, and thus, for example, a rechargeable battery (not shown) is provided in the casing 12.

The casing 12 is a chest piece of the electronic stethoscope 10, and has a first end surface 12a that comes into contact with an organism when being used, a second end surface 12b that is situated on a side opposite to the first end surface 12a, and an outer peripheral surface 12c that connects the first end surface 12a and the second end surface 12b to each other. The casing 12 when viewed in a facing direction in which the first end surface 12a and the second end surface 12b face each other (the Z axis direction) has a circular shape. It should be noted that, in the first embodiment, the casing 12 has a shape whose outside diameter decreases in a direction away from the first end surface 12a and from the second end surface 12b, that is, the casing 12 has a shape including a narrow portion.

In the first embodiment, as shown in FIGS. 2 and 3, the casing 12 includes a top case 20 including the first end surface 12a, a middle case 22 including a portion of the outer peripheral surface 12c, a bottom case 24 including a portion of the outer peripheral surface 12c, and a fixing ring 26 including the second end surface 12b and fixing the diaphragm 14 to the bottom case 24.

Further, in the first embodiment, as shown in FIG. 1, a display 28 and a plurality of buttons 30A to 30D are provided on the second end surface 12b of the casing 12.

The diaphragm 14 is a flexible sheet member made of an elastic material. The diaphragm 14 is provided at the first end surface 12a of the casing 12 so as to be disposed to face an organism when the electronic stethoscope 10 is being used. In the first embodiment, an outer peripheral edge of the diaphragm 14 is fixed to the bottom case 24 through the fixing ring 26.

When the first end surface 12a of the casing 12 comes into contact with an organism, the diaphragm 14 vibrates with a frequency and an amplitude based on the sound of the organism (for example, a heart sound) that is produced by the organism.

As shown in FIG. 3, the sound sensor 16 is provided in the bottom case 24 of the casing 12, and receives vibration (that is, the sound of the organism) propagated from the diaphragm 14 and converts the vibration into an electrical signal (organism sound data). The sound sensor 16 is, for example, a microphone.

As shown in FIG. 3, the circuit board 18 is provided in the middle case 22 of the casing 12. As shown in FIG. 4, for example, a processor 30, a storage device 32, and a wireless communication module 34 are provided on the circuit board 18. The processor 30 is, for example, a CPU or an MPU that is mounted on the circuit board 18. The storage device 32 is, for example, a memory that is mounted on the circuit board 18 or a memory card that is attachably and detachably inserted into the electronic stethoscope 10. The wireless communication module 34 is a device that is mounted on the circuit board 18 and that performs wireless communication with an external device (not shown) by a wireless communication method that is in conformity with a predetermined wireless communication standard such as Bluetooth (registered trademark).

The processor 30 of the electronic stethoscope 10 performs various operations in accordance with a program that is stored in the storage device 32. For example, the processor 30 causes the display 28 to display an operation state of the electronic stethoscope 10. For example, the processor 30 converts an electrical signal that is outputted from the sound sensor 16 into organism sound data. The processor 30 causes the storage device 32 to store the organism sound data. The organism sound data is sent to, for example, an external device (not shown) through the wireless communication module 34. It should be noted that the operations of the processor 30 are not limited to these operations.

In the first embodiment, the processor 30 of the electronic stethoscope 10 has a structure that calculates an S/N ratio of the sound of an organism that the sound sensor 16 has obtained through the diaphragm 14. The processor 30 has a structure that, based on the calculated S/N ratio, determines the sound quality of the obtained sound of the organism, and notifies a user of a determination result thereof.

Specifically, the electronic stethoscope 10 has a structure that by light notifies the user of the determination result of the sound quality of the sound of the organism. Therefore, as shown in FIG. 3, the electronic stethoscope 10 includes a plurality of light-emitting elements 36A to 36C and a light-guiding member 38.

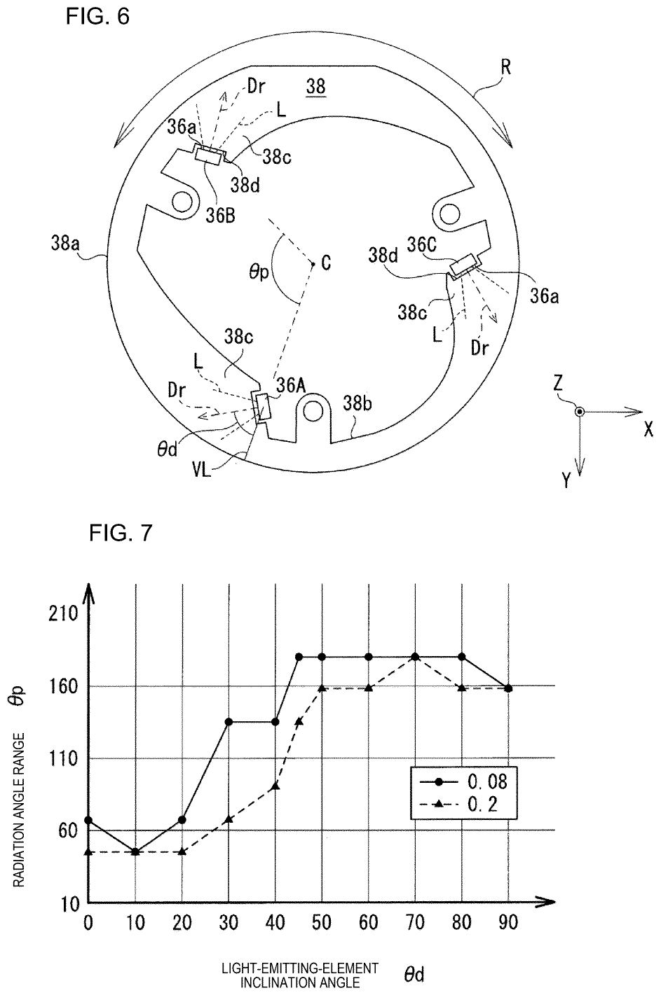

FIG. 5 is a perspective view of the light-emitting elements and the light-guiding member provided on the circuit board in the electronic stethoscope according to the first embodiment. FIG. 6 is a cross-sectional view of the light-emitting elements and the light-guiding member in the electronic stethoscope according to the first embodiment. It should be noted that in order to make it easier to read FIG. 6, the light-emitting elements and the light-guiding member are not hatched.

As shown in FIG. 5, in the first embodiment, a plurality of light-emitting elements 36A to 36C are, for example, LEDs, and are provided on the circuit board 18 and thus are provided in the casing 12. It should be noted that, in the first embodiment, the plurality of light-emitting elements 36A to 36C are the same light-emitting elements.

In the first embodiment, the light-emitting elements 36A to 36C are each a so-called sidelight-type light-emitting element that emits a light beam L along a surface of the circuit board 18, and each has a planar light-emitting surface 36a. As shown in FIG. 6, the light-emitting surfaces 36a of the respective light-emitting elements 36A to 36C face an outer side of the casing 12 when viewed in the facing direction in which the first end surface 12a and the second end surface 12b of the casing 12 face each other (the Z axis direction).

Further, in the first embodiment, the light-emitting elements 36A to 36C each have a structure that is capable of emitting light of a plurality of colors. For example, the light-emitting elements 36A to 36C each include a red LED, a green LED, and a blue LED. This makes it possible to notify a user of the sound quality of the sound of an organism based on color differences. When the sound quality of the sound of the organism is good, such as the sound having little noise, the light-emitting elements 36A to 36C each emit green light. In addition, for example, when the sound quality of the sound of the organism is not good, such as the sound having a lot of noise, the light-emitting elements 36A to 36C each emit yellow light. Further, for example, when the sound volume level of the sound of the organism is low, the light-emitting elements 36A to 36C each emit orange light.

The light-guiding member 38 is a member for guiding to the outside of the casing 12 the light beams L of the respective light-emitting elements 36A to 36C provided in the casing 12. The light-guiding member 38 is made of a light-transmissive resin material, such as polycarbonate or acrylic.

In the first embodiment, as shown in FIGS. 5 and 6, the light-guiding member 38 when viewed in the facing direction in which the first end surface 12a and the second end surface 12b of the casing 12 face each other (the Z axis direction) is a ring-shaped member having an outer peripheral surface (outer surface) 38a that faces the outside of the casing 12 and an inner peripheral surface (inner surface) 38b that faces the inside of the casing 12. In particular, in the first embodiment, the light-guiding member 38 has a substantially circular ring shape. The light-guiding member 38 is provided on the circuit board 18 so as to surround the plurality of light-emitting elements 36A to 36C on the circuit board 18.

As shown in FIGS. 1 and 2, the outer peripheral surface 38a of the light-guiding member 38 is an exposure portion that is exposed to the outside of the casing 12. The outer peripheral surface 38a of the light-guiding member 38 when viewed in the facing direction in which the first end surface 12a and the second end surface 12b of the casing 12 face each other (the Z axis direction) extends along the outer peripheral surface 12c of the casing 12.

As shown in FIG. 6, the light-guiding member 38 includes a plurality of light-receiving portions 38c that protrude toward the light-emitting surfaces 36a of the respective light-emitting elements 36A to 36C from the inner peripheral surface 38b thereof and that face the respective light-emitting surfaces 36a. That is, the light beams L of the respective light-emitting elements 36A to 36C are each incident upon a corresponding one of the light-receiving portions 38c. Therefore, the light-emitting elements 36A to 36C each do not face the outer peripheral surface 38a of the light-guiding member 38, and the light beams L thereof are not incident upon the light-guiding member 38 through the outer peripheral surface 38a.

In the first embodiment, each of the light-receiving portions 38c has an incident surface 38d that is parallel to a corresponding one of the light-emitting surfaces 36a. It should be noted that, in the first embodiment, since the light-emitting surfaces 36a of the respective light-emitting elements 36A to 36C have planar shapes, the incident surfaces 38d also have planar shapes; however, the embodiment of the present disclosure does not limit the shape of each incident surface 38d. For example, when the light-emitting elements each have a hemispherical convex-shaped light-emitting surface, the incident surfaces of the light-guiding member may each have a hemispherical concave shape.

In the first embodiment, as shown in FIG. 6, the plurality of light-emitting elements 36A to 36C when viewed in the facing direction in which the first end surface 12a and the second end surface 12b of the casing 12 face each other (the Z axis direction) are provided on the circuit board 18 at a substantially constant interval (an angular interval of approximately 120 degrees) along a peripheral direction R of the circular ring-shaped light-guiding member 38. Therefore, the corresponding light-receiving portions 38c are also arranged side by side at the substantially constant interval along the peripheral direction R.

When the light-emitting surfaces 36a of the respective light-emitting elements 36A to 36C emit the light beams L, the light beams L while spreading are incident upon the incident surfaces 38d of the respective light-receiving portions 38c. From the incident surfaces 38d, the light beams L start propagating in the light-guiding member 38. The light beams L while being reflected several times in the light-guiding member 38 are finally emitted to the outside of the light-guiding member 38 through the outer peripheral surface 38a of the light-guiding member 38. Therefore, the light beams L are radiated toward the outside of the casing 12 from the entire outer peripheral surface 38a of the light-guiding member 38. As a result, the entire outer peripheral surface 38a of the light-guiding member 38 emits light and the light emission is visually recognized by a user.

Orientations in which the light-emitting elements 36A to 36C are disposed with respect to the respective light-receiving portions 38c of the light-guiding member 38 are determined such that the entire outer peripheral surface 38a of the light-guiding member 38 emits light with a substantially uniform luminance, that is, such that the intensities of the light beams L that are emitted to the outside become substantially equal to each other over the entire outer peripheral surface 38a of the light-guiding member 38.

Specifically, in the first embodiment, the light-emitting elements 36A to 36C are provided with respect to the light-receiving portions 38c of the light-guiding member 38 such that emission directions Dr of the light beams L of the respective light-emitting elements 36A to 36C intersect nonorthogonally the outer peripheral surface 38a of the light-guiding member 38. More specifically, as shown in FIG. 6, the emission directions Dr of the respective light-emitting elements 36A to 36C are inclined at predetermined inclinations angle θd with respect to imaginary straight lines VL that connect the respective light-emitting elements 36A to 36C and the outer peripheral surface 38a of the light-guiding member 38 to each other at the shortest distances. It should be noted that FIG. 6 illustrates the imaginary straight line VL and the predetermined inclination angle θd with respect to the light-emitting element 36A, and does not illustrate the imaginary straight lines VL and the predetermined inclination angles θd with respect to the respective light-emitting elements 36B and 36C.

Here, in the present specification, the “emission direction” of a light-emitting element refers to a direction in which, of a plurality of light rays that are emitted in various directions in a predetermined angle range from the light-emitting element, a light ray having the highest luminance propagates. For example, when a light beam of a light-emitting element is projected onto a screen, a direction of extension of a straight line that connects the light-emitting element and a portion of a projected image appearing on the screen with the highest luminance corresponds to the “emission direction” of the light-emitting element. It should be noted that, in the first embodiment, since the light-emitting surfaces 36a of the respective light-emitting elements 36A to 36C have planar shapes, each “emission direction” is a direction normal to the corresponding light-emitting surface 36a.

FIG. 7 illustrates relationships between an inclination angle of a light-emitting element and a radiation angle range of light from the outer peripheral surface of the light-guiding member. It should be noted that a radiation angle range θp shown in FIG. 7 is an angle range that starts from an angular position of the light-emitting element as shown in FIG. 6.

As shown in FIG. 7, as long as the inclination angle θd of the light-emitting element is an angle in a range of 30 to 90 degrees, light having a luminance ratio of 0.08 is radiated from the outer peripheral surface 38a of the light-guiding member 38 in the radiation angle range θp that is approximately 130 degrees. As long as the inclination angle θd of the light-emitting element is an angle in a range of 45 to 80 degrees, light having a luminance ratio of 0.2 is radiated from the outer peripheral surface 38a of the light-guiding member 38 in the radiation angle range θp that is approximately 130 degrees. It should be noted that the “luminance ratio” refers to a ratio to the luminance of light of the light-emitting element before the light is incident upon the light-guiding member.

That is, as long as the inclination angles θd of the respective light-emitting elements 36A to 36C are angles in the range of 30 to 90 degrees (more specifically, angles in the range of 45 to 80 degrees), the light beams L emitted therefrom are reflected more times and are propagated farther in the light-guiding member 38. In addition, when the three light-emitting elements 36A to 36C having such inclination angles θd are disposed at an angular interval of approximately 120 degrees as shown in FIG. 6, the entire outer peripheral surface 38a of the light-guiding member 38 can emit light with a luminance ratio of 0.08 (preferably, 0.2).

It should be noted that when the inclination angles θd of the light-emitting elements 36A to 36C are less than 30 degrees, the radiation angle ranges Op become less than 120 degrees. This is because, when large quantities of the light beams emitted from the light-emitting elements 36A to 36C first reach the outer peripheral surface 38a of the light-guiding member 38, they are emitted without being reflected. In this case, portions of the outer peripheral surface 38a of the light-guiding member 38 near the light-emitting elements 36A to 36C emit light with higher luminances than other portions. Therefore, in this case, in order to cause the entire outer peripheral surface 38a of the light-guiding member 38 to emit light with a luminance ratio of 0.08 (preferably, 0.2), four or more light-emitting elements are required.

According to the first embodiment described above, the electronic stethoscope where the light-emitting elements emit light for notifying a user of predetermined information can reliably notify the user of the predetermined information by using a small number of light-emitting elements.

Specifically, the light beams of the three light-emitting elements 36A to 36C can be radiated to the outside of the casing 12 in a radiation angle range of 360 degrees through the outer peripheral surface 38a of the endless light-guiding member 38 extending along the entire outer peripheral surface 12c of the casing 12 when viewed in the facing direction in which the first end surface 12a and the second end surface 12b of the casing 12 face each other (the Z axis direction). Therefore, even if a user covers a portion of the outer peripheral surface 38a of the light-guiding member 38 with any of his fingers, the user can visually recognize the light emission of the light-guiding member 38. As a result, the user can reliably obtain information based on the light emission.

Further, incident surfaces 38d of the respective light-receiving portions 38c of the light-guiding member 38 are parallel to the light-emitting surfaces 36a of the respective light-emitting elements 36A to 36C. Therefore, the light beams L of the light-emitting elements 36A to 36C are orthogonally incident upon the incident surfaces 38d. As a result, the light beams of the light-emitting elements 36A to 36C can be efficiently taken into the light-guiding member 38.

As a result, it is possible to reliably inform the user of the predetermined information by using the light beams of a small number of light-emitting elements.

As a secondary effect, it is possible to keep low power consumption of the light-emitting elements because the light beams of the small number of light-emitting elements 36A to 36C are radiated to the outside of the casing 12 in the radiation angle range of 360 degrees through the light-guiding member 38. Further, since the number of light-emitting elements is small, a smaller space is needed for disposing the light-emitting elements, as a result of which it is possible to decrease the size of the entire electronic stethoscope 10, or to increase the degree of freedom of disposing other structural elements.

### Second Embodiment

A second embodiment is an improvement of the above-described first embodiment, and is specifically a form that increases the luminance of light that is radiated from an outer peripheral surface of a light-guiding member. Therefore, the light-guiding member of the second embodiment differs from the light-guiding member of the above-described first embodiment. It should be noted that structural elements of the second embodiment that are substantially the same as the structural elements of the above-described first embodiment are given the same reference numerals.

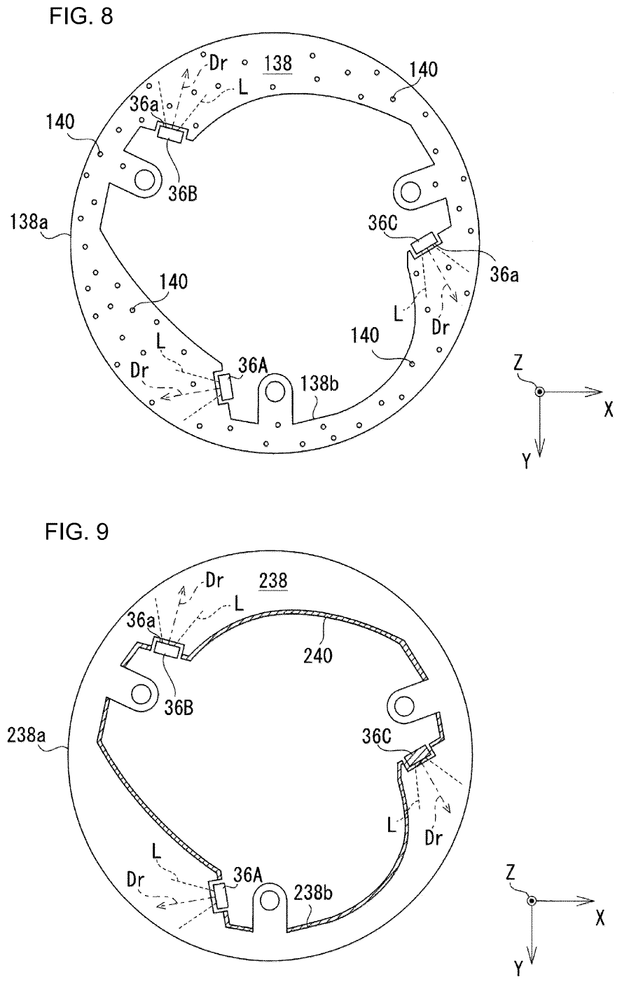

FIG. 8 is a cross-sectional view of light-emitting elements and the light-guiding member in an electronic stethoscope according to the second embodiment. It should be noted that in order to make it easier to read FIG. 8, the light-emitting elements and the light-guiding member are not hatched.

As shown in FIG. 8, in the electronic stethoscope according to the second embodiment, a plurality of diffusing members 140 for diffusing light beams L of light-emitting elements 36A to 36C are dispersed in a light-guiding member 138. The diffusing members 140 cause the light beams L to reflect randomly in the light-guiding member 138. Due to the random reflection of the light beams L, the light-beams L can propagate in the light-guiding member 138 up to locations that are far away from the respective light-emitting elements 36A to 36C. As a result, an entire outer peripheral surface 138a of the light-guiding member 138 emits light with a more substantially uniform luminance. That is, light emission unevenness of the outer peripheral surface 138a is suppressed.

Even in the second embodiment described above, as in the above-described first embodiment, the electronic stethoscope where the light-emitting elements emit light for notifying a user of predetermined information can reliably notify the user of the predetermined information by using a small number of light-emitting elements.

### Third Embodiment

A third embodiment is an improvement of the above-described first embodiment, and is specifically a form that increases the luminance of light that is radiated from an outer peripheral surface of a light-guiding member. Therefore, the light-guiding member of the third embodiment differs from the light-guiding member of the above-described first embodiment. It should be noted that structural elements of the third embodiment that are substantially the same as the structural elements of the above-described first embodiment are given the same reference numerals.

FIG. 9 is a cross-sectional view of light-emitting elements and the light-guiding member in an electronic stethoscope according to the third embodiment. It should be noted that in order to make it easier to read FIG. 9, the light-emitting elements and the light-guiding member are not hatched.

As shown in FIG. 9, in the electronic stethoscope according to the third embodiment, a reflecting layer 240, such as silver plating or an aluminum film, that reflects light is provided at at least a part of a surface portion other than an outer peripheral surface 238a of a light-guiding member 238, which is an exposure portion exposed to the outside of a casing, the part being, for example, an inner peripheral surface 238b. It should be noted that the material of the reflecting layer 240 is to be a material having high reflectivity, and examples of the material of the reflecting layer 240 include silver, gold, aluminum, and stainless steel. Light beams L that are emitted from respective light-emitting elements 36A to 36C and that propagate in the light-guiding member 238 are reflected by the reflecting layer 240, and are finally emitted through the outer peripheral surface 238a. That is, the light beams L that propagate through the light-guiding member 238 are such that emission of the light beams L from a surface portion where the reflecting layer 240 is provided is suppressed. As a result, the light quantity of the light beams L that are emitted from the outer peripheral surface 138a of the light-guiding member 138 is further increased, and the outer peripheral surface 238a emits the light with a higher luminance.

Even in the third embodiment described above, as in the above-described first embodiment, the electronic stethoscope where the light-emitting elements emit light for notifying a user of predetermined information can reliably notify the user of the predetermined information by using a small number of light-emitting elements.

### Fourth Embodiment

A fourth embodiment is an improvement of the above-described first embodiment, and is specifically a form that increases the luminance of light that is radiated from an outer peripheral surface of a light-guiding member. Therefore, the light-guiding member of the fourth embodiment differs from the light-guiding member of the above-described first embodiment. It should be noted that structural elements of the fourth embodiment that are substantially the same as the structural elements of the above-described first embodiment are given the same reference numerals.

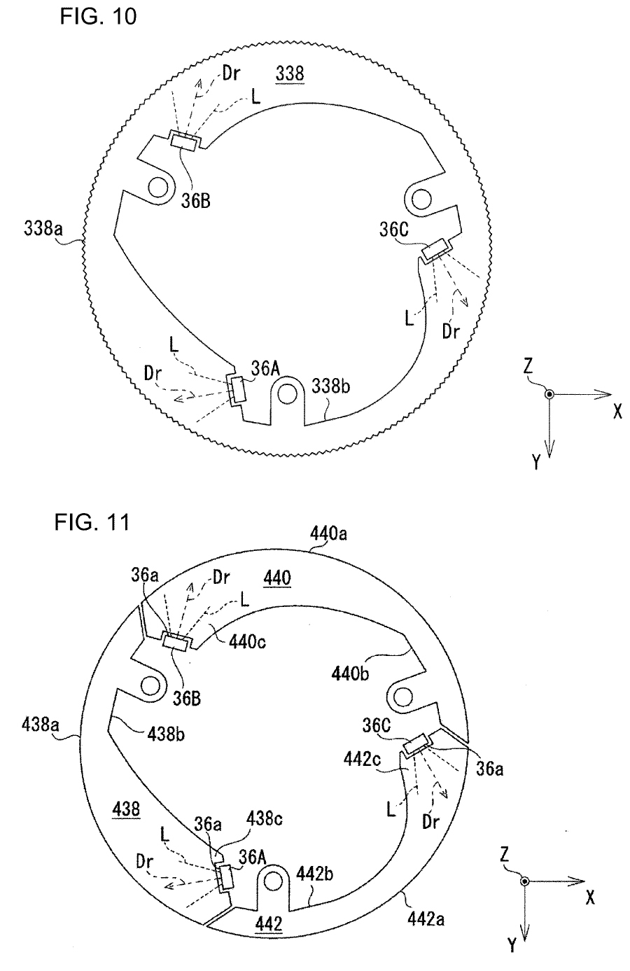

FIG. 10 is a cross-sectional view of light-emitting elements and the light-guiding member in an electronic stethoscope according to the fourth embodiment. It should be noted that in order to make it easier to read FIG. 10, the light-emitting elements and the light-guiding member are not hatched.

As shown in FIG. 10, in the electronic stethoscope according to the fourth embodiment, an outer peripheral surface 338a of a light-guiding member 338, which is an exposure portion exposed to the outside of a casing, is rougher than other surface portions. For example, the outer peripheral surface 338a is a periodically or a nonperiodically uneven surface. According to the outer peripheral surface 338a that has been made rough in this way, light beams L that are emitted from respective light-emitting elements 36A to 36C and that propagate in the light-guiding member 238 are unlikely to be reflected by the outer peripheral surface 338a, but are likely to be emitted from the outer peripheral surface 338a. As a result, the light quantity of the light beams L that are emitted from the outer peripheral surface 338a of the light-guiding member 338 is further increased.

It should be noted that, when the outer peripheral surface 338a is made rough, it is preferable that surface portions of the light-guiding member 338 other than the outer peripheral surface 338a be given a mirror finish (for example, be provided with a reflecting layer as in the above-described third embodiment). This makes it possible for the light beams L that propagate in the light-guiding member 338 where the possibility of reflection at the outer peripheral surface 338a has been reduced to be reflected by surfaces other than the outer peripheral surface 338a and thus to be capable of propagating even farther in the light-guiding member 338.

For example, the outer peripheral surface 338a of the light-guiding member 338 has a surface roughness (arithmetic average roughness) Ra that is greater than or equal to 1.6 μm, and the surface portions other than the outer peripheral surface 338a has a surface roughness Ra in a range of 0.8 to 1.6 μm. For example, when the light-guiding member 338 is a resin molded product, the entire light-guiding member 338 after the molding has a surface roughness Ra that is substantially less than or equal to 1.6 μm. By roughening, such as sandblasting, the outer peripheral surface 338a of the light-guiding member 338 after the molding, the outer peripheral surface 333a can have a surface roughness that is greater than or equal to 1.8 μm, such as 3.0 μm. As methods of measuring the surface roughness Ra, there exist a contact-type measuring method using a stylus that, while contacting a measurement surface, moves along the surface, and a non-contact-type measuring method that scans a measurement surface with laser. It should be noted that the present disclosure is not limited to the method of measuring the surface roughness Ra.

Even in the fourth embodiment described above, as in the above-described first embodiment, the electronic stethoscope where the light-emitting elements emit light for notifying a user of predetermined information can reliably notify the user of the predetermined information by using a small number of light-emitting elements.

Although the present disclosure has been described by way of a plurality of embodiments, the embodiments of the present disclosure are not limited thereto.

For example, in the above-described first embodiment, the light-emitting elements 36A to 36C emit light beams for notifying a user of the quality of a biological sound obtained by the sound sensor 16, as a result of which the outer peripheral surface 38a of the light-guiding member 38 is caused to emit light. However, in the embodiment of the present disclosure, information of which the user is notified due to the light emission by the outer peripheral surface of the light-guiding member is not limited thereto. For example, when the electronic stethoscope is provided with a rechargeable battery, for notifying the user that the battery is fully charged, the outer peripheral surface of the light-guiding member may emit light during the charging.

In the above-described first embodiment, the light-guiding member 38 is a substantially circular ring-shaped member. However, the embodiment of the present disclosure is not limited thereto. For example, the light-guiding member may be a polygonal ring-shaped member. The light-guiding member of the electronic stethoscope is not limited to having a ring shape and is not limited to one in number.

FIG. 11 is a cross-sectional view of light-emitting elements and light-guiding members in an electronic stethoscope according to a different embodiment. It should be noted that in order to make it easier to read FIG. 11, the light-emitting elements and the light-guiding members are not hatched.

As shown in FIG. 11, the electronic stethoscope according to the different embodiment includes three light-guiding members 438, 440, and 442. Light-emitting elements 36A, 36B, and 36C are each provided at a corresponding one of the light-guiding members 438, 440, and 442.

The light-guiding members 438, 440, and 442 when viewed in a facing direction in which a first end surface and a second end surface of a casing face each other (the Z axis direction) are arc-shaped and are disposed on the same circumference. The light-guiding members 438, 440, and 442 each include a corresponding one of outer surfaces 438a, 440a, and 442a, which are exposure portions that are exposed to the outside of the casing, and a corresponding one of inner surfaces 438b, 440b, and 442b. Further, the inner surfaces 438b, 440b, and 442b of the respective light-guiding members 438, 440, and 442 each include a corresponding one of light-receiving portions 438c, 440c, and 442c that protrude toward light-emitting surfaces 36a of the respective light-emitting elements 36A, 36B, and 36C and that face the respective light-emitting surfaces 36a. The light-emitting elements 36A, 36B, and 36C are disposed with respect to the light-receiving portions 438c, 440c, and 442c of the respective light-guiding members 430, 440, and 442 such that their emission directions Dr nonorthogonally intersect the exposure portions (the outer surfaces 438a, 440a, and 440a) of the respective light-guiding members 430, 440, and 442.

Similarly to the light-guiding member 38 of the above-described first embodiment, even such plurality of light-guiding members 438, 440, and 442 can radiate light beams of the respective light-emitting elements 36A to 36C in a radiation angle range of 360 degrees to the outside of the casing. When any one of the plurality of light-emitting elements 36A to 36C emits a light beam L to a corresponding one of the light-guiding members and thus an outer peripheral surface of the light-guiding member emits the light beam, it is possible to notify a user of directional information. For example, it is possible to notify the user of information regarding a direction of existence of a sound source (such as a direction of existence of the heart) of the sound of an organism with respect to the electronic stethoscope.

Further, in the above-described first embodiment, the electronic stethoscope 10 includes three light-emitting elements, the light-emitting elements 36A to 36C. However, the embodiment of the present disclosure is not limited thereto. When the light-emitting elements have high directivity and/or emit light beams having high luminance, the number of light-emitting elements that are provided in the electronic stethoscope may be two or less.

Further, in the above-described first embodiment, the plurality of light-emitting elements 36A to 36C each include a red LED, a green LED, and a blue LED. Therefore, the plurality of light-emitting elements 36A to 36C notify a user of the sound quality of the sound of an organism based on color differences. However, the light emission colors of the light-emitting elements may be changed due to reasons other than notifying the user of the sound quality of the sound of the organism.

For example, the light-emitting elements may emit light beams of particular light emission colors with respect to a particular organism. For example, when an organism to be auscultated with the electronic stethoscope is an infant, the light-emitting elements may emit light beams of warm colors that an infant likes, such as yellow or orange.

With regard to an organism being an infant, the infant sometimes does not like light itself. In this case, the light from the electronic stethoscope may be prevented from being directed toward the face of the infant.

For example, in the above-described first embodiment, one or some of the plurality of light-emitting elements 36A to 36C emit light beams, and the remaining one or ones of the light-emitting elements stop emitting light beams. Then, a user uses the electronic stethoscope 10 with respect to the infant such that the light emitted from the electronic stethoscope 10 does not strike the face of the infant.

It should be noted that, when all of the light-emitting elements 36A to 36C emit light beams, only the light beams that are directed toward the face of the infant are blocked.

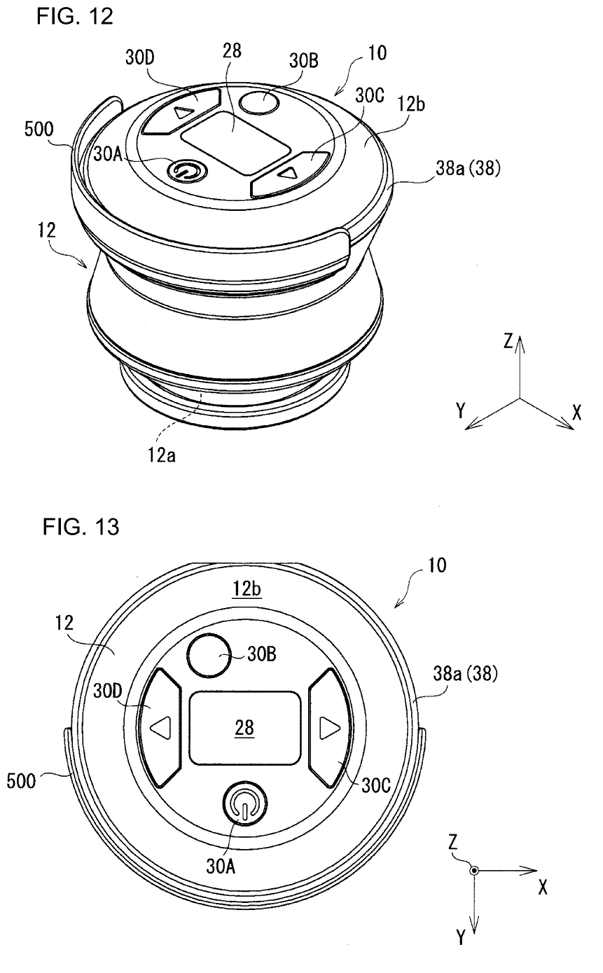

FIGS. 12 and 13 are, respectively, a perspective view and a top view of the electronic stethoscope according to the first embodiment with a light-shielding cover being attached thereto.

As shown in FIGS. 12 and 13, a light-shielding cover 500 that covers a portion of the outer peripheral surface 38a of the light-guiding member 38 is attached to the electronic stethoscope 10. The light-shielding cover 500 is a substantially semicircular cylindrical member that, while extending in the facing direction in which the first end surface 12a and the second end surface 12b of the casing 12 face each other (the Z axis direction), extends along the portion of the outer peripheral surface 38a of the light-guiding member 38. The light-shielding cover 500 is non-transparent, and is made of, for example, a resin material.

According to such a light-shielding cover 500, a part of light emitted from the portion of the outer peripheral surface 38a of the light-guiding member 38 covered by the light-shielding cover 500 is blocked by the light-shielding cover 500. Specifically, light beams emitted in directions (the X axis direction, the Y axis direction) that intersect the facing direction in which the first end surface 12a and the second end surface 12b of the casing 12 face each other (the Z axis direction) are blocked by the light-shielding cover 500. Therefore, when the user places the electronic stethoscope 10 on, for example, the chest of an infant such that the light-shielding cover 500 exists near the head of the infant, the light that is directed toward the face of the infant from the electronic stethoscope 10 is blocked by the light-shielding cover 500.

It should be noted that, as shown in FIG. 12, the outer peripheral surface 38a of the light-guiding member 38 of the electronic stethoscope 10 when viewed in the directions (the X axis direction, the Y axis direction) that intersect the facing direction in which the first end surface 12a and the second end surface 12b of the casing 12 face each other (the Z axis direction) is a convex curved surface on an outer side. Therefore, the light that is emitted in the facing direction from such outer peripheral surface 38a is not blocked by the light-shielding cover 500. Consequently, a user that uses the electronic stethoscope 10 with respect to an infant can visually recognize the light from the portion of the outer peripheral surface 38a of the light-guiding member 38 that is covered by the light-shielding cover 500.

It should be noted that the light-shielding cover 500 may be attachably and detachably provided at the casing 12 or the light-guiding member 38. Alternatively, the light-shielding cover 500 may be provided at the casing so as to be capable of advancing toward and withdrawing from the casing 12, and may be stored in the casing 12 when not being used.

As described above, when light emission colors with respect to an infant are to be changed and/or light emissions of some of the light-emitting elements are to be stopped, the electronic stethoscope may have an infant mode. For example, in the electronic stethoscope 10 according to the first embodiment, a user executes the infant mode by performing a predetermined operation on any one of the plurality of buttons 30A to 30D, and the light emission colors of the light-emitting elements 36A to 36C are changed to warm colors and/or the light emission of any one or any two of the light-emitting elements 36A to 36C is stopped.

With regard to the light emissions of the light-emitting elements, the light-emitting elements may emit light beams with a luminosity suitable for the use environment of the electronic stethoscope. For example, when the light-emitting elements emit light beams with a luminosity suitable for the illuminance of a consulting room of a medical facility, the light-emitting elements emit light beams with a luminosity that is calculated based on Numerical Expressions 1 and 2 below. For example, according to Japanese Industrial Standard (JIS), the illuminance of a consulting room of an insurance medical facility is determined as being 300 to 750 lx (lux).

$$\begin{matrix}[ Numerical Expression 1 ] & \\ B = \frac{L \times R}{π} & ( Numerical Expression 1 )\end{matrix}$$

$$\begin{matrix}[ Numerical Expression 2 ] & \\ B = \frac{I}{S} & ( Numerical Expression 2 )\end{matrix}$$

In Numerical Expressions 1 and 2, L is the illuminance (lx) of the use environment of the electronic stethoscope, R is the reflectivity of the outer peripheral surface of the light-guiding member, S is the area (m²) of the outer peripheral surface of the light-guiding member, B is the luminance (cd/m²) of light that is radiated from the light-guiding member, and I is the luminosity (cd) of the light-emitting elements. According to Numerical Expressions 1 and 2, if the illuminance L of the use environment of the electronic stethoscope is determined, the luminosity I of the light-emitting elements is univocally determined.

The luminosity of the light-emitting elements may be adjustable by a user. For example, in the electronic stethoscope 10 according to the above-described first embodiment, the electronic stethoscope 10 may have a structure that allows a user to adjust the luminosity of the light-emitting elements 36A to 36C by performing a predetermined operation on any one of the plurality of buttons 30A to 30D. In this case, the luminosity adjustment range of the light-emitting elements 36A to 36C is set to a range corresponding to an illuminance range of 300 to 750 Lx.

Alternatively, based on the brightness of the use environment of the electronic stethoscope, the luminosity of the light-emitting elements may be changed. In this case, the electronic stethoscope includes an illuminance sensor that detects the brightness (illuminance) of the surroundings thereof, and a processor changes the luminosity of the light-emitting elements based on a detection signal from the illuminance sensor.

Finally, in the above-described first embodiment, as shown in FIG. 6, the light beams L from the respective light-emitting elements 36A to 36C are orthogonally incident upon the incident surfaces 36d of the respective light-receiving portions 36c of the light-guiding member 38. However, the embodiment of the present disclosure is not limited thereto. That is, as long as light beams that start propagating in the light-guiding member from the light-receiving portions propagate through the light-guiding member in directions that nonorthogonally intersect the exposure portion of the light-guiding member when viewed in the facing direction in which the first end surface and the second end surface of the casing face each other (the Z axis direction), the incidence angles of the light beams of the light-emitting elements with respect to the light-guiding member do not matter. This is because, when, unlike the above, light beams that start propagating in the light-guiding member from the light-receiving portions propagate in directions orthogonal to the exposure portion of the light-guiding member, the light beams exit to the outside of the light-guiding member as they are through the exposure portion. In this case, the exposure portion of the light-guiding member cannot emit light over the entire extension direction thereof.

That is, in a broad sense, the embodiment of the present disclosure is an electronic stethoscope including a casing that has a first end surface that faces an organism when being used, a second end surface that is situated on a side opposite to the first end surface, and an outer peripheral surface that connects the first end surface and the second end surface to each other; a sound sensor that is provided at the casing and that obtains a sound of the organism and converts the sound of the organism into an electrical signal; a light-emitting element that is provided in the casing and that has a light-emitting surface that emits light; and a light-guiding member that guides the light of the light-emitting element to outside of the casing, in which the light-guiding member has an inner surface that includes a light-receiving portion that faces the light-emitting surface of the light-emitting element and upon which the light of the light-emitting element is incident, and an outer surface that is exposed to the outside of the casing by extending along the outer peripheral surface of the casing when viewed in a facing direction in which the first end surface and the second end surface face each other, and that radiates to the outside of the casing the light that is incident upon the outer surface through the light-receiving portion.

### REFERENCE SIGNS LIST

36A: light-emitting element
36B: light-emitting element
36C: light-emitting element
36a: light-emitting surface
38: light-guiding member
38a: outer surface (outer peripheral surface)
38c: light-receiving portion
L: light beam

1. An electronic stethoscope comprising:
a casing that has a first end surface that is adapted to face an organism when being used, a second end surface that is situated on a side opposite to the first end surface, and an outer peripheral surface that connects the first end surface to the second end surface;
a sound sensor that is provided at the casing and that is adapted to obtain the a sound of the organism and to convert the sound of the organism into an electrical signal;
a light-emitting element that is provided in the casing and that has a light-emitting surface that emits light; and
a light-guiding member that guides the light of the light-emitting element to outside of the casing,
wherein the light-guiding member has
an inner surface that includes a light-receiving portion that faces the light-emitting surface of the light-emitting element and upon which the light of the light-emitting element is incident, and
an outer surface that is exposed to the outside of the casing by extending along the outer peripheral surface of the casing when viewed in a facing direction in which the first end surface and the second end surface face each other, and that radiates to the outside of the casing the light that is incident upon the outer surface through the light-receiving portion.

2. The electronic stethoscope according to claim 1,
wherein the light-guiding member has a ring shape when viewed in the facing direction.

3. The electronic stethoscope according to claim 1,
wherein a plurality of the light-emitting elements are disposed at a plurality of locations, and
wherein the light-guiding member includes a plurality of the light-receiving portions at the inner surface in correspondence with the plurality of the light-emitting elements.

4. The electronic stethoscope according to claim 1,
wherein the light-receiving portion of the light-guiding member has an incident surface that is parallel to the light-emitting surface of the light-emitting element and upon which the light of the light-emitting element is orthogonally incident, and
wherein, when viewed in the facing direction, an angle between an emission direction of the light-emitting element and an imaginary straight line that connects the light-emitting element and the exposure portion of the light-guiding member to each other at a shortest distance is between 30 to 90 degrees.

5. The electronic stethoscope according to claim 4,
wherein, when viewed in the facing direction, the angle between the imaginary straight line and the emission direction of the light-emitting element is between 45 to 80 degrees.

6. The electronic stethoscope according to claim 1, further comprising:
a circuit board on which the light-guiding member and the light-emitting element are provided, and
wherein the light-emitting element is a sidelight-type light-emitting element.

7. The electronic stethoscope according to claim 1,
wherein the light-emitting element is adapted to emit light of a plurality of colors.

8. The electronic stethoscope according to claim 1, further comprising:
a processor that controls light emission of the light-emitting element based on the electrical signal that has been output from the sound sensor.

9. The electronic stethoscope according to claim 1, wherein a plurality of diffusing members that diffuse light are dispersed in the light-guiding member.

10. The electronic stethoscope according to claim 1,
wherein a reflecting layer is provided at at least a part of a surface portion other than the exposure portion of the light-guiding member.

11. The electronic stethoscope according to claim 1,
wherein a surface of the exposure portion of the light-guiding member is rougher than other surface portion of the light-guiding member.

12. The electronic stethoscope according to claim 1, further comprising:
a light-shielding cover that partly covers the outer surface of the light-guiding member.

13. The electronic stethoscope according to claim 12,
wherein the light-shielding cover blocks light that is radiated from the outer surface of the light-guiding member in a direction that intersects the facing direction.
